(19) United States
(12) Patent Application Publication
(10) Pub. No.: US 2011/0311532 A1
(43) Pub. Date: Dec. 22, 2011

(54) INHIBITION OF COMPLEMENT AND CELLULAR ACTIVATION

(76) Inventor: Rekha Bansal, Twinsburg, OH (US)

(21) Appl. No.: 12/675,906

(22) PCT Filed: Aug. 29, 2008
(86) PCT No.: PCT/US08/74779

§ 371 Date: Apr. 29, 2011

Related U.S. Application Data
(60) Provisional application No. 60/968,681, filed on Aug. 29, 2007.

(51) Int. Cl. A61K 39/395; A61P 37/06; A61P 1/16; A61P 9/00; A61P 29/00; A61P 7/00; A61P 35/00; A61P 31/18

(52) U.S. Cl. 424/134.1; 424/141.1; 424/130.1; 424/133.1

(57) ABSTRACT

A method of inhibiting Fc mediated platelet activation in a subject induced by administration of a therapeutic antibody or fragment thereof includes administering to the subject an amount of anti-platelet agent effective to inhibit Fc induced platelet activation in the subject.

### RELATED APPLICATION

This application claims priority from U.S. Provisional Application No. 60/968,681, filed Aug. 29, 2007, the subject matter, which is incorporated herein by reference.

### FIELD OF THE INVENTION

The present invention relates to a method of mitigating complications that result from administration of therapeutic IgG isoforms that have an Fc region to a subject, and particularly relates to a method of inhibiting aberrant platelet activation that results from administration of IgG isoforms that have an Fc region to treat therapeutic disorders, such as cancer or complement mediated disorders.

### BACKGROUND

Complement activation plays a major role in tissue injury due to the release of pro-inflammatory molecules from this process. Complement can be activated through either of three distinct enzymatic cascades, referred to as the “classical”, Lectin and “alternative” pathways (CP, MBL, and AP respectively). Activated complement species, particularly the anaphylatoxins, C3a and C5a, are known to elicit a variety of inflammatory responses from many cell types. For example, C3a is known to activate monocytes, and initiate cytokine, prostaglandin, and histamine release. It also plays a significant role in cognition and asthma. Likewise, C5a is known to up-regulate cell adhesion molecule expression on neutrophils, invoke lysosomal enzyme (elastase), free radical release from neutrophils, monocytes, and activate platelets. Platelet activation by anaphylatoxins contributes to abnormal bleeding, such as those seen in post cardiopulmonary bypass (CBP) operations, by preventing normal clotting function. Finally, the terminal activated complement product, C5b-9 (also known as ‘membrane-attack complex’), can also affect platelet function. It is the actions of these complement species on neutrophils, platelets, monocytes, and other circulatory cells that likely lead to the various problems that arise after CPB.

The bioactive molecules produced after the activation of either complement pathway include the anaphylatoxins, C3a and C5a, as well as the terminal complement complex known as C5b-9 or the membrane-attack complex (MAC). The anaphylatoxins initiate a cellular inflammatory response that can be beneficial in the case of a pathogenic infection, but are detrimental when they are inappropriately generated. Neutrophils, monocytes, and platelets have been known to express C3a and C5a receptors. Upon C3a and C5a receptor activation, these cells release molecules that cause tissue damage. Both neutrophils and monocytes express CD11b on the cell surface, and activated platelets express CD62P. The activated neutrophils and monocytes bind platelets to form leukocyte-platelet conjugates that are well known to be markers of complement-mediated diseases. Inhibition of platelet activation should also prevent the formation of leukocyte-platelet conjugates

Various types of complement inhibitors, both small molecules such as compstatin and macromolecules such as anti-factors P, B, D, and C5 antibodies have been tested for blocking complement activation in whole human blood. Some studies presented data on cellular activation while others do not. An example of a peptide-based complement inhibitor is compstatin. This peptide binds to C3, C3b, and C3c and is known to inhibit C3a, C5a and C5b-9 generation in vitro, and in vivo. Small molecule inhibitors of factor D and C3a/C5a receptor antagonists have also been investigated with limited success. Both C3a and C5a receptors have been found on platelets, monocytes, and neutrophils. Therefore, inhibition of C3a and C5a production should downregulate cellular activation.

Monoclonal antibodies used as a therapeutic to prevent cancer proliferation have an acute Fc region with upregulated ADCC activity. As a result, cancer killing is increased via the Fc region. As an innocent bystander, platelets are attacked as well because of the Fc receptors on platelets. In such cases, cancer patients suffer from bleeding complications clot formation and other platelet mediated effects including platelet-leukocyte conjugates during and after the treatment.

### SUMMARY

The present invention relates to a method of treating a disorder in a subject. The method includes administering to a subject a therapeutic agent that can treat the disorder and an anti-platelet agent. The therapeutic agent induces Fc mediated activation of platelets in the subject, and the anti-platelet agent is effective to inhibit Fc mediated platelet activation.

In an aspect of the invention, the therapeutic agent can include a monoclonal antibody of the IgG isotype or an antibody fragment comprising an Fc region. The IgG can include at least one of IgG1, IgG2, IgG3, or IgG4. The therapeutic agent can also include at least one of a monoclonal antibody, polyclonal antibody, protein including sequences of Fc, and fusion protein including sequences of Fc.

The therapeutic agent can be used to treat, for example, at least one of cancer, allograft rejection, HIV, cardiopulmonary bypass, rheumatoid arthritis, stroke, multiple sclerosis, sepsis, autoimmune hematological disorders, complement mediated disorders, hepatitis B. In one example, the therapeutic agent can be used to treat cancer. In another example, the therapeutic agent can be used to treat a complement mediated disorder.

In another aspect, the anti-platelet agent can include a compound that binds to or modulates the GPIIb/IIIa receptor present on platelets. The compound can be a GPIIb/IIIa receptor antagonist that inhibits platelet aggregation. The anti-platelet agent can inhibit platelet aggregation, leukocyte-platelet aggregation, and platelet CD62P expression.

Alternatively, the anti-platelet agent can inhibit platelet aggregation without inhibiting platelet CD62P expression. The anti-platelet agent can also inhibit IgG platelet activation.

In a further aspect, the anti-platelet agent can include at least one of a COX 1 inhibitor, a COX 2 inhibitor, ADP receptor antagonist, GPIIb/IIIa inhibitor, or phosphodiesterase inhibitor.

The GPIIb/IIIa inhibitor can be selected from the group consisting of abciximab, eptifibatide, tirofiban, lamifiban, lefradafiban, sibrafiban (Ro-48-3657), orbofiban, xemilofiban, and combinations thereof. In a specific example, the anti-platelet agent can be aspirin or abciximab.

In a further aspect of the invention, the administration of the therapeutic agent and the anti-platelet agent does not inhibit activation of leukocytes and NK cells. Alternatively, the administration of the therapeutic agent and the antiplatelet agent inhibits activation of leukocytes, NK cells, neutrophils, eosinophils, platelets, B-cells, dendritic cells, and endothelial cells.

The present invention also relates to a method of treating a cancer in a subject. The method includes administering to a subject a therapeutic antibody or fragment thereof comprising an Fc sequence and an anti-platelet agent. The therapeutic antibody or fragment thereof can treat cancer and induce Fc mediated activation of platelets in the subject. The amount of an anti-platelet agent administered to the subject is effective to inhibit platelet activation.

In an aspect of the invention, the therapeutic antibody can include a monoclonal antibody of the IgG1 isotype and the anti-platelet agent can include a compound that binds to or modulates the GPIIb/IIIa receptor present on platelets. The compound can include a GPIIb/IIIa receptor antagonist that inhibits platelet aggregation. The anti-platelet agent can inhibit platelet aggregation without inhibiting platelet CD62P expression. The anti-platelet agent can include at least one of a COX 1 inhibitor, a COX 2 inhibitor, ADP receptor antagonist, GPIIb/IIIa inhibitor, or phosphodiesterase inhibitor. The GPIIb/IIIa inhibitor can be selected from the group consisting of abciximab, eptifibatide, tirofiban, lamifiban, lefradafiban, sibrafiban (Ro-48-3657), orbofiban, xemilofiban, and combinations thereof. The administration of the therapeutic agent and the anti-platelet agent does not inhibit activation of leukocytes and NK cells.

The present invention further relates to a method of treating a complement mediated disorder in a subject. The method includes administering to a subject a therapeutic antibody or fragment thereof comprising an Fc sequence and an anti-platelet agent. The therapeutic antibody or fragment thereof can inhibit complement activation and induce Fc mediated activation of platelets in the subject. The anti-platelet agent can be administered at amount effective to inhibit platelet activation.

In aspect of the invention, the therapeutic antibody can include a monoclonal antibody of the IgG isotype or an antibody fragment and the anti-platelet agent can include a compound that binds to or modulates the GPIIb/IIIa receptor present on platelets. The compound can include a GPIIb/IIIa receptor antagonist that inhibits platelet aggregation. The anti-platelet agent can inhibit platelet aggregation without inhibiting platelet CD62P expression. The anti-platelet agent can include at least one of a COX 1 inhibitor, a COX 2 inhibitor, ADP receptor antagonist, GPIIb/IIIa inhibitor, or phosphodiesterase inhibitor. The GPIIb/IIIa inhibitor can be selected from the group consisting of abciximab, eptifibatide, tirofiban, lamifiban, lefradafiban, sibrafiban (Ro-48-3657), orbofiban, xemilofiban, and combinations thereof.

In another aspect, the anti-platelet can inhibit platelet aggregation, leukocyte-platelet aggregation, and platelet CD62P expression. In a further aspect, the administration of the therapeutic antibody or protein and the anti-platelet agent does not inhibit activation of leukocytes and NK cells. Alternatively, administration of the therapeutic antibody or fragment thereof and the antiplatelet agent inhibits activation of leukocytes, NK cells, neutrophils, eosinophils, platelets, B-cells, dendritic cells, and endothelial cells.

The present invention further relates to a method of inhibiting a Fc mediated platelet activation in a subject induced by administration of a therapeutic antibody or fragment thereof. The method includes administering to the subject an amount of anti-platelet agent effective to inhibit Fc induced platelet activation in the subject.

In an aspect of the invention, the therapeutic antibody can include a monoclonal antibody of the IgG isotype or an antibody fragment comprising an Fc region. The therapeutic antibody can also include at least one of a monoclonal antibody, polyclonal antibody, protein including sequences of Fc, and fusion protein including sequences of Fc.

The therapeutic antibody or fragment thereof can be used to treat at least one of cancer, allograft rejection, HIV, cardiopulmonary bypass, rheumatoid arthritis, stroke, multiple sclerosis, sepsis, autoimmune hematological disorders, complement mediated disorders, hepatitis B. In one example, the therapeutic antibody or fragment thereof can be used to treat cancer. In another example, the therapeutic antibody or fragment thereof can be used to treat a complement mediated disorder.

In another aspect, the anti-platelet agent can include a compound that binds to or modulates the GPIIb/IIIa receptor present on platelets. The compound can be a GPIIb/IIIa receptor antagonist that inhibits platelet aggregation. The anti-platelet agent can inhibit platelet aggregation, leukocyte-platelet aggregation, and platelet CD62P expression. Alternatively, the anti-platelet agent inhibits platelet aggregation without inhibiting platelet CD62P expression. The anti-platelet agent can also inhibit IgG platelet activation.

In a further aspect, the anti-platelet agent can include at least one of a COX 1 inhibitor, a COX 2 inhibitor, ADP receptor antagonist, GPIIb/IIIa inhibitor, or phosphodiesterase inhibitor.

The GPIIb/IIIa inhibitor can be selected from the group consisting of abciximab, eptifibatide, tirofiban, lamifiban, lefradafiban, sibrafiban (Ro-48-3657), orbofiban, xemilofiban, and combinations thereof. In a specific example, the anti-platelet agent can be aspirin or abciximab.

In a further aspect of the invention, the administration of the anti-platelet agent does not inhibit activation of leukocytes and NK cells. Alternatively, the administration of the antiplatelet agent inhibits activation of leukocytes, NK cells, neutrophils, eosinophils, platelets, B-cells, dendritic cells, and endothelial cells.

### BRIEF DESCRIPTION OF THE DRAWINGS

FIG. 1 shows two panels; the top panel shows graphs for C3a, C5a, and sC5b-9 measurements in ELISA. The bottom panel is for neutrophils, monocytes, and platelets. The first column in each panel is a control sample. This control sample is from the blood filled tubing loop that has been rotated at 37 degree for 2 hours. The columns 2 to 7 are the same as the first one but with the added NM4047. Columns 2, 4, and 6 contain 400 μg/ml of NM4047 whereas the columns 3, 5, and 7 contain 100 μg/ml of NM4047. All blood samples after circulation were centrifuged and evaluated for C3a, C5a, and sC5b-9 using ELISA shown in top panel and for neutrophil, monocyte and platelet activation using flow cytometry. Neutrophil and monocyte activation was measured using CD11b and platelet activation was measured using CD62P labels. The figure shows that NM4047 inhibits complement but not cellular activation.

FIG. 2 shows two panels; the top panel is for complement and the bottom panel is for flow cytometry results. Intact IgG1 (NM4047) was fragmented by Ficin to remove the Fc region. As shown, the inhibition of C3a, C5a, and sC5b-9 is consistent with intact NM4047. Surprisingly activation of all three cell types was completely abolished. These data suggest that the activation was mediated via Fc. IgG1 is known to activate the Fc shown in table 1, in fact all three cell types are activated. While this activation is not desired for some therapies, it is required for others.

FIG. 3 shows two panels; the top panel is for complement and the bottom panel is for cellular activation. Blood samples were mixed with 400 μg/ml and 100 μg/ml of NM4047 as shown in the figure. All samples contain the same amount of ReoPro (a monoclonal antibody Fab, 10 μg/ml) to demonstrate if Fc effects on platelets can also be inhibited by the GPIIbIIIa antagonist ReoPro. As shown, ReoPro does not inhibit neutrophil and monocyte activation. It only inhibits platelet activation to a nearly 100% level. These data clearly indicate the new finding that Reopro can inhibit NM4047 induced activation of platelets. Such combinations will be useful to selectively inhibit the undesired activation of platelets but maintain the desired activation of leukocytes for effective therapy. One such example is the cancer therapy in which activation of NK cells/and monocytes is desired for effective killing of cancer cells.

FIG. 4 illustrates the effect of ReoPro on NM4047 mediated activation of platelets. The data in FIG. 3 was extended only for platelets in FIG. 4. Blood samples containing 400 μg/ml concentration of NM4047 were incubated with various concentrations of ReoPro ranging from 0.5 to 50 μg/ml of ReoPro. All samples other than controls contain 400 μg/ml of Nm4047. The effect of ReoPro were dose dependent with ReoPro being effective at a concentration of between 0.5 to 1 μg/ml. These concentrations provide much significant inhibition of platelet activation.

FIG. 5 illustrates the effect of aggrastat, a different GPIIbIIIa antagonist small molecule on inhibition of platelet activation. Similar to the experiment in FIG. 4, Aggrastat at various concentrations was added to blood containing 400 μg/ml of NM4047 (IgG1). Aggrastat concentration ranged from 1 μg/ml to 20 μg/ml. All samples other than controls contain 400 μg/ml of NM4047. As shown, Aggrastat appears to be highly potent in inhibiting the Fc mediated activation of platelets. Aggrastat had no effect on neutrophils or monocytes. Therefore, it seems to be similar to ReoPro in its action on platelet activation.

FIG. 6 illustrates the effect of Integrillin, a cyclic peptide” which blocks platelet aggregation but not platelet activation as measured by the CD62P staining. The experiment was primarily set up similar to the ones described in FIGS. 4 and 5. Integrillin was used in a dose dependent manner. As shown, Integrillin activated platelets in a dose dependent fashion with highest concentration showing higher platelet activation. All samples other than controls contain 400 μg/ml of NM4047. It is known that Integrillin does not inhibit platelet-leukocyte interactions and therefore its use is limited to certain cardiac procedures. It is not clear if the aggravated platelet activation seen in these experiments are due to the Fc mediated responses from NM4047 conjugated with known platelet activation by integrillin. The total activation by integrillin was at least 4 fold at 100 μg/ml. Obviously human dose currently used in patients is much lower.

FIG. 7 illustrates the effect of Aspirin in combination with NM4047 IgG1 on the activation of platelets. All samples other than controls contain 400 μg/ml of NM4047. Aspirin has only partial effect on neutrophils and monocytes. As shown on platelets, aspirin inhibits platelet activation in this combination therapy. Aspirin effect on platelets was not as robust as observed for ReoPro and Aggrastat makes it less likely for clinical situation. The use of aspirin suggest that other class of molecules can be identified to serve as a combination agent. Aspirin was tested at 5-50 μg/ml concentration. All samples other than controls contain 400 μg/ml of NM4047.

FIG. 8 shows the predicted effect of a blocking complement IgG2 antibody on complement and cellular activation in whole blood. All complement activationmarkers will be inhibited at a given concentration which need to be determined using methods well known in the art. In the bottom panel are shown activation of neutrophils, monocytes and platelets. As shown in Table-1, IgG1 binds all three cell types among others. ReoPro only inhibits platelet activation. IgG2 binds and activated only platelets and does not bind neutrophils and monocytes. ReoPro or the selected GPIIb/IIa antagonist can inhibit platelet activation. Therefore, an ideal blocking monoclonal antibody should be an IgG2 we predict the effect of such an antibody would be as shown in FIG. 8. IgG3 will activate neutrophils and monocytes. IgG4 is also a likely choice but could activate monocytes.

### DETAILED DESCRIPTION

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab', F(ab′)2, Fv, single-chain antibodies, truncated antibodies and F(ab′)2 truncated antibodies. An antibody other than a “bispecific” or “bifunctional” antibody is understood to have each of its binding sites identical.

The term “monoclonal” refers to an antibody that binds to a sequence of amino acid and has a single specific epitope on its target antigen.

The term “polyclonal” refers to an antibody that recognizes multiple epitope sites on a single antigen.

The term “epitope” includes any protein determinant capable of specific binding to an immunoglobulin. Epitope determinants usually consist of chemically active surface groupings of molecules such as amino acids or sugar side chains and usually have specific three-dimensional structural characteristics, as well as specific charge characteristics.

The terms “oligomer” and “polymer” are used interchangeable. The terms “oligomer” and “polymer” refer to the association of more than one monomer of a specific protein, peptide, or peptide fragments. The terms “oligomer” and “polymer” in this invention specifically relates to the ability of properdin protein monomers to form protein complexes with it or with other proteins.

The term “agent” is used herein to denote a chemical compound, a mixture of chemical compounds, a biological macromolecule, or an extract made from biological materials.

The terms “patient”, “mammalian host”, “subject”, “mammalian subject” and the like are used interchangeably herein, and refer to mammals, including human and veterinary subjects.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

The term “a complement mediated disorder,” as used herein, refers to a disease or disorder caused, directly or indirectly, by activation of the complement pathway, a disease or disorder that is mediated, directly or indirectly, by one or more components of the complement pathway, or a product generated by the complement pathway. The term also refers to a disease or disorder that is exacerbated by one or more components of the complement pathway, or a product generated by the complement pathway.

It is to be understood that this invention is not limited to particular embodiments described. It is also to be understood that the terminology used herein is for describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are encompassed within the invention, subject to any specifically excluded limit in the stated range. Where the stated range includes one or both of the limits, ranges excluding either or both of those included limits are also included in the invention.

The present invention relates to methods of inhibiting Fc induced or mediated platelet activation using an anti-platelet agent. Mouse or human antibodies contain an active Fc region based on the antibody type. In certain disease conditions, such as cancer, the Fc region of the antibody can be used for more efficient killing of cancer cells. While an upregulated Fc can be required for cancer therapy, the Fc region of the antibody can activate platelets via the Fc receptor. Activated platelets are responsible for platelet-mediated inflammatory effects such as leukocyte-platelet conjugates, which can lead to tissue factor expression on monocytes and could cause thrombosis events. In addition, activated platelets can form platelet aggregates with fibrinogen and can cause clot formation. We have found that an anti-platelet agent can prevent Fc-mediated platelet activation. These findings are used to generate combination therapies where antibodies, fragments thereof that include the Fc region, or proteins that include the Fc region are used as therapeutics with anti-platelet agents.

One aspect of the present invention therefore relates to a method of treating a disorder that includes administering to a subject a therapeutic agent, which can treat the disorder and which induces Fc mediated activation of platelets in the subject, in combination with an anti-platelet agent. The disease or disorder treated by the therapeutic agent can include, for example, cancer, atherosclerosis, ischemia-reperfusion following acute myocardial infarction, Henoch-Schonlein purpura nephritis, immune complex vasculitis, rheumatoid arthritis, arteritis, aneurysm, stroke, cardiomyopathy, hemorrhagic shock, crush injury, multiple organ failure, hypovolemic shock and intestinal ischemia, transplant rejection, cardiac Surgery, PTCA, spontaneous abortion, neuronal injury, spinal cord injury, myasthenia gravis, Huntington's disease, amyotrophic lateral sclerosis, multiple sclerosis, Guillain Bane syndrome, Parkinson's disease, Alzheimer's disease, acute respiratory distress syndrome, asthma, chronic obstructive pulmonary disease, transfusion-related acute lung injury, acute lung injury, Goodpasture's disease, myocardial infarction, post-cardiopulmonary bypass inflammation, cardiopulmonary bypass, septic shock, transplant rejection, xeno transplantation, burn injury, systemic lupus erythematosus, membranous nephritis, Berger's disease, psoriasis, pemphigoid, dermatomyositis, anti-phospholipid syndrome, inflammatory bowel disease, hemodialysis, leukopheresis, plasmapheresis, heparin-induced extracorporeal membrane oxygenation LDL precipitation, extracorporeal membrane oxygenation, macular degeneration, and combinations thereof. In a specific example, the disorder can include cancer, such as colon cancer, breast cancer, lung cancer, and ovarian cancer.

The therapeutic agent that induces a Fc mediated platelet activation can include an antibody with an epitope that is recognized by the Fc receptors and activates platelets (e.g., Fc region), an antibody fragment with an epitope that is recognized by the Fc receptors and activates platelets (e.g., Fc region), a protein or fustion protein with an epitope that is recognized by the Fc receptors and activates platelets (e.g., includes an amino acid sequence of an Fc region or a mimetic of the Fc region), and small molecule with an epitope that is recognized by the Fc receptors and activates platelets.

In one example, the therapeutic agent can include a monoclonal antibody of the IgG isotype or an antibody fragment comprising an Fc region. The IgG can include at least one of IgG1, IgG2, IgG3, or IgG4.

IgG isotypes can activate various cells including monocytes, neutrophils, macrophages, eosinophils, platelets, B cells, dendritic cells, endothelial cells, and subsets of T cells. The particular cells activated by the respective isotypes are shown in the following table.

TABLE
Characterization of IgG Isoforms

| | IgG1 | IgG2 | IgG3 | IgG4 |
|---|---|---|---|---|
| Adult serum level range | 4.9-11.4 | 1.5-6.4 | 0.20-1.10 | 0.08-1.40 |
| (g/l) | | | | |
| Proportion of total IgG (%) | 43-75 | 16-48 | 1.7-7.5 | 0.8-11.7 |
| Half-life (days) | 21 | 21 | 7 | 21 |
| FCgRI (CD64: monocytes, | ++ | − | +++ | + |
| neutrophils) | | | | |
| FcGRII (CD32): | ++ | − | +++ | − |
| monocytes, macrophages, | | | | |
| neutrophils, eosinophils, | | | | |
| platelets, B cells, | | | | |
| dendritic cells, endothelial | | | | |
| cells) | | | | |
| FcGRIIa-H131 | ++ | +++ | +++ | − |
| FcGRIIa-R131 | ++ | − | ++ | − |
| FcGRIII (CD16: | ++ | − | ++ | − |
| neutrophils, eosinophils, | | | | |
| macrophages, NK | | | | |
| cells, subsets of T cells) | | | | |
| FcGRIIIb-NA1 | +++ | − | +++ | − |
| FcGRIIIb-NA2 | ++ | − | ++ | − |

Examples of therapeutic agents that are monoclonal antibodies that can be administered in combination with the ant-platelet agent are 5G1.1, a humanised IgG antibody, which is produced by Alexion Pharm Inc. and is used to treat Rheumatoid Arthritis, Systemic Lupus Erythematosus (SLE), and Nephritis; ABX-CBL, a human IgG antibody, which is produced by Abgenix, Inc. and is used to treat Graft Versus Host Disease (GVHD) and Allograft rejection; ABX-IL8, a human IgG2 antibody, which is produced by Abgenix, Inc. and is used to treat Psoriasis; AD-159, a humanised IgG antibody, which is produced by Tanox Biosystems and is used to treat Human Immunodeficiency Virus (HIV); AD-439, a humanised IgG antibody, which is produced by Tanox Biosystems and is used to treat Human Immunodeficiency Virus (HIV); Antegren, a humanised IgG antibody, which is produced by Athena/Elan and is used to treat Multiple Sclerosis; Anti-CD11a, a humanised IgG1 antibody, which is produced by Genentech, Inc. and is used to treat Psoriasis; Anti-VEGF, a humanised IgG1 antibody, which is produced by Genetech, Inc. and is used to treat Cancer; Antova, a humanised IgG antibody, which is produced by Biogen and is used to treat Allograft rejection and Systemic Lupus Erythematosus (SLE); BEC2, a Murine IgG antibody, which is produced by ImClone Sys/Merk KGaA and is used to treat Lung disorders; BIRR-1, a Murine IgG2a antibody, which is produced by Boehringer Pharm and is used to treat Strokes; BTI-322, a Rat IgG antibody, which is produced by Abgenix, Inc. and is used to treat Graft Versus Host Disease (GVHD); C225, a Chimeric IgG antibody, which is produced by Imclone sys and is use d to treat Head and Neck disorders; CAT-152, a human IgG antibody, which is produced by Cambridge Ab Tech and is used to treat Glaucoma; CFP571, a humanised IgG4 antibody, which is produced by Celltech and is used to treat Crohn's Disease and Rheumatoid Arthritis; CDP850, a humanised IgG antibody, which is produced by Celltech and is used to treat Psoriasis; Corsevin M, a Chimeric IgG antibody, which is produced by Centocor and is used to treat Anticoagulant; D2E7, a human IgG antibody, which is produced by CAT/BASF and is used to treat Rheumatoid Arthritis; Herceptin, a humanised IgG1 antibody, which is produced by Genetech and is used to treat Metastatic Breast; HNK20, a Murine IgA antibody, which is produced by Peptide Therap Gp and is used to treat Respiratory Syncytial Virus (RSV); Hu23F2G, a humanised IgG antibody, which is produced by ICOS Pharm, Inc. and is used to treat Multiple Sclerosis and stroke; IC14, a humanised IgG antibody, which is produced by ICOS Pharm, Inc. and is used to treat Toxic Shock; ICM13, a humanised IgG antibody, which is produced by ICOS Pharm, Inc. and is used to treat Psoriasis; IDEC-114, a Primatised IgG antibody, which is produced by IDEC Pharm/Mitsubishi and is used to treat Psoriasis; IDEC-131, a humanised IgG antibody, which is produced by IDEC Pharm/Eisai and is used to treat Systemic Lupus Erythematosus (SLE) and Multiple Sclerosis; IDEC-151, a Primatised IgG1 antibody, which is produced by IDEC Pharm/GlaxoKlineSmith and is used to treat Rheumatoid Arthritis; IDEC-152, a Primatised IgG antibody, which is produced by IDEC Pharm and is used to treat Asthma/Allergy; Infliximab, a Chimeric IgG1 antibody, which is produced by Centocor and is used to treat Rheumatoid Arthritis and Crohn's Disease; LPD-01, a humanised IgG antibody, which is produced by Millennium Inc. (LeukoSite Inc.) and is used to treat Stroke and Allograft Rejection; LDP-02, a humanised IgG antibody, which is produced by Millennium, Inc. (LeukoSite Inc.) and is used to treat Ulcerative Colitis; LDP-03/Campath1H, a humanised IgG1 antibody, which is produced by Millennium, Inc. (LeukoSite Inc.) and is used to treat Chronic Lymphocytic Leukemia (CLL); Lym-1, a Chimeric IgG antibody, which is produced by Techiclone Corp. and is used to treat Non-Hodgkin's lymphoma (NHL); LympoCide, a humanised IgG antibody, which is produced by Immonomedics and is used to treat Non-Hodgkin's lymphoma (NHL); MDX-33, a human IgG antibody, which is produced by Medarex/Centeon and is used to Autoimmune haematogical disorders; MDX-CD4, a human IgG antibody, which is produced by Medarex/Eisai/Genmab and is used to treat Rheumatoid Arthritis; OKT4A, a humanised IgG antibody, which is produced by Ortho Biotech and is used to treat Allograft rejection; OrthoClone OKT4A, a humanised IgG antibody, which is produced by Ortho Biotech and is used to treat Autoimmune Disease; Orthoclone/anti-CD3 OKT3, a Murine mIgG2a antibody, which is produced by Ortho Biotech and is used to treat Allograft rejection; Ostavir, a human IgG antibody, which is produced by Protein Design Lab/Novartis and is used to treat Hepatitis B; OvaRex, a Murine IgG antibody, which is produced by Altarex and is used to treat Ovarian; Panorex 17-1A, a Murine IgG2a antibody, which is produced by GlaxoSmithKline/Centocor and is used to treat Colorectal; PRO542, a humanised IgG antibody, which is produced by Progenics/Genzyme Transgenics and is used to treat Human Immunodeficiency Virus (HIV); Protovir, a humanised IgG1 antibody, which is produced by Prot Design Lab/Novartis and is used to treat Cytomegalovirus (CMV); rhuMab-E25, a humanised IgG1 antibody, which is produced by Genentech/Novartis/Tanox Biosystems and is used to treat Asthma/Allergy Rituxan, a Chimeric IgG1 antibody, which is produced by IDEC Pharm and is used to treat Non-Hodgkin's lymphoma (NHL); SB-240563, a humanised IgG antibody, which is produced by GlaxoSmithKline and is used to treat Asthma/Allergy; SB-240683, a humanised IgG antibody, which is produced by GlaxoSmithKline and is used to treat Asthma/Allergy; SCH-55700, a humanised IgG antibody, which is produced by GlaxoSmithKline and is used to treat Asthma/Allergy; Simulect, a Chimeric IgG1 antibody, which is produced by Novartis Pharm and is used to treat Allograft rejection; SMART a-CD3, a humanised IgG antibody, which is produced by Protein Design Lab and is used to treat Autoimmune Disease, Allograft rejection, and Psoriasis; SMART-M195, a humanised IgG antibody, which is produced by Protein Design Lab/Kanebo and is used to treat Acute Myeloid Leukemia (AML); SMART-1D10, a humanised IgG antibody, which is produced by Protein Design Lab and is used to treat Non-Hodgkin's Leukemia (NHL); Synagis, a humanised IgG1 antibody, which is produced by MedImmune and is used to Respiratory Syncytial Virus (RSV) (pediatric); Vitaxin, a humanised IgG antibody, which is produced by Ixsys and is used to treat Sarcoma; and Zenapax, a humanised IgG1 antibody, which is produced by Protein Design Lab/Hoffman-La Rouche and is used to treat Allograft Rejection.

The anti-platelet agent can include any agent that inhibits Fc mediated platelet activation and/or aggregation. For the purpose of this invention, the phrase, “platelet activation,” is defined as a process that leads to platelet CD62P expression, platelet-leukocyte conjugate formation, or other platelet activities that result in platelet dysfunction. Thus, agents that can inhibit activation of platelets or obviate platelet dysfunction are classified as anti-platelet agents.

There are several classes of platelet activation inhibitors and each has different mechanisms of inhibiting platelet activation. The four major classes currently used in clinical practice are COX-1 and COX-2 inhibitors, ADP receptor antagonists, GPIIb/IIIa inhibitors, and phosphor-diesterase inhibitors.

COX-1 and COX-2 inhibitors inhibit platelet activation by targeting the enzymes and inhibiting their activity. By blocking the functions of COX-1 and COX-2 enzymes, the production of prostaglandins and thromboxanes—key molecules that regulate platelet activation and aggregation—are inhibited. Aspirin is currently used as a blood-thinner in clinical practice and acts by binding irreversibly to COX-1 and modifying the function of COX-2 enzymes.

ADP receptor antagonists function by binding to or altering the functionality of platelet ADP receptors. By blocking platelet ADP receptors, the interaction between ADP and the platelet receptor P2Y(12) is disrupted. Two currently marketed drugs, Ticlopidine and Clopidogrel fall under this class and irreversibly affect ADP binding to P2Y(12) hence disrupting platelet activation of the GPIIb/IIIa receptor and thus platelet activation and aggregation. Another two compounds, Cangrelor and AZD6140, also target the P2Y(12) receptor but their inhibition is reversible. The end result is the inhibition of platelet activation and aggregation.

Glycoprotein IIb/IIIa receptor is expressed following agonist stimulation. This receptor binds with multiple adhesive ligand molecules, including fibrinogen, vWF (in conditions of high shear as might exist in stenotic arteries), fibronectin, vitronectin, and thrombospondin, which cause platelets to aggregate. This receptor activation is the final step in the platelet activation cascade. Regardless of the activation mechanism, expression of the GPIIb/IIIa receptor is the end result. Blockers of GPIIb/IIIa activity readily inhibit platelet activation and aggregation.

Three drugs, Abciximab (ReoPro), Eptifibatide (Integrilin), and Tirofiban (Aggrastat), are currently being used in clinical practice for the inhibition of platelet activation via the GPIIb/IIIa mechanism. Abcixmab is a monoclonal F(ab) fragment GPIIb/IIIa antagonist that binds to the GPIIb/IIIa receptor and remains bound for extended periods of time. Integrillin is a cyclic heptapeptide that also binds to the GPIIb/IIIa receptor and inhibits its activity. It is noted for its short half-life making it an ideal candidate for acute administration. Aggrastat also targets the GPIIb/IIIa receptor but alters the conformation of the receptor making it inactive. The benefit of this molecule is that it is a small molecule with short clearance rate making it ideal for acute indications.

The last major class of platelet activation inhibitors are the phosphodiesterase inhibitors. These drug targets function by targeting the PDE3, which is responsible for inhibiting second messenger production of cAMP and cGMP and thus inhibiting the NO/cGMP signaling pathway, which is primarily responsible for the activation of platelets. Current drugs on the market that target this enzyme include cilastazol, Piroximone, Enoximone, Toborinone, Olprinone, and Dipyridamole.

In an aspect of the invention, the anti-platelet agent can include a compound that binds to or modulates the GPIIb/IIIa receptor present on platelets. The compound can be a GPIIb/IIIa receptor antagonist that inhibits platelet aggregation. The anti-platelet agent can inhibit platelet aggregation, leukocyte-platelet aggregation, and platelet CD62P expression. Alternatively, the anti-platelet agent can inhibit platelet aggregation without inhibiting platelet CD62P expression.

In a further aspect, the anti-platelet agent can include a GPIIb/IIIa inhibitor and be selected from the group consisting of abciximab, eptifibatide, tirofiban, lamifiban, lefradafiban, sibrafiban (Ro-48-3657), orbofiban, xemilofiban, and combinations thereof. In a specific example, the anti-platelet agent can be aspirin or abciximab.

The therapeutic agent and the anti-platelet agent can be administered to the subject in a therapeutically effective amount. A therapeutically effective amount of the therapeutic agent and the anti-platelet agent of the present invention is readily determinable by one of ordinary skill in the art, and is dependent upon the type and severity of clinical condition. The therapeutic agent and the anti-platelet agent may be administered to the patient in a clinically acceptable manner such as intravenous, intraperitoneal, or subcutaneous injection or infusion; oral administration; transdermal absorption through the skin; topical administration; or inhalation. The dosage may vary according to the clinical procedure contemplated, generally ranging from about 1 nanomolar to about 100 millimolar. The therapeutic agent and the anti-platelet agent may be administered simultaneously or sequentially. If the therapeutic agent and the anti-platelet agent are to be administered sequentially, then it must be done so sufficiently closely in time so as to provide the desired therapeutic effect.

Pharmaceutical compositions comprising the therapeutic agent and the anti-platelet agent can be administered to humans and other mammals enterally or parenterally in a solid, liquid, or vapor form. Enteral route includes oral, rectal, topical, buccal, and vaginal administration. Parenteral route includes intravenous, intramuscular, intraperitoneal, intrasternal, and subcutaneous injection or infusion. The compositions can also be delivered through a catheter for local delivery at a target site, via an intracoronary stent (a tubular device composed of a fine wire mesh), or via a biodegradable polymer.

The therapeutic agent and the anti-platelet agent of the present invention may be mixed under sterile conditions with a pharmaceutically acceptable carrier along with any needed, but physiologically tolerable, preservatives, excipients, buffers, propellants, diluents, carriers, adjuvants, and the like. The phrase “pharmaceutically acceptable” means those formulations which are, within the scope of sound medical judgment, suitable for use in contact with the tissues of humans and animals without undue toxicity, irritation, allergic response and the like, and are commensurate with a reasonable benefit/risk ratio. A comprehensive discussion on the types and uses of various buffers, excipients, diluents, carriers, adjuvants, and the like for a pharmaceutically acceptable compositions are provided in “Remington: The Science and Practice of Pharmacy”.

Actual dosage levels of the active ingredients in the pharmaceutical formulation can be varied so as to achieve the desired therapeutic response for a particular patient. The selected dosage level will depend upon the activity of the particular compound, the route of administration, the severity of the condition being treated, and prior medical history of the patient being treated. However, it is within the skill of the art to start doses of the compound at levels lower than required to achieve the desired therapeutic effect and to increase it gradually until optimal therapeutic effect is achieved. The total daily dose of the compounds of this invention administered to a human or lower animal may range from about 0.0001 to about 1000 mg/kg/day. For purposes of oral administration, more preferable doses can be in the range from about 0.001 to about 5 mg/kg/day. If desired, the effective daily dose can be divided into multiple doses for purposes of administration; consequently, single dose compositions may contain such amounts or submultiples thereof to make up the daily dose.

The therapeutically effective amount of the therapeutic agent and the anti-platelet agent of the invention can include a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated, the severity of the disorder; activity of the specific compound employed; the specific composition employed, age, body weight, general health, sex, diet of the patient; the time of administration, route of administration, and rate of excretion of the specific compound employed, and the duration of the treatment. The compounds of the present invention may also be administered in combination with other drugs if medically necessary.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Prolonged absorption of the injectable pharmaceutical form can be brought about by the use of agents delaying absorption, for example, aluminum monostearate and gelatin. Proper fluidity can be maintained, for example, by the use of coating materials such as lecithin, by the maintenance of the required particle size in the case of dispersions, and by the use of surfactants. In some cases, in order to prolong the effect of the drug, it is desirable to slow the absorption of the drug. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissues. The injectable formulations can be sterilized, for example, by filtration through a bacterial-retaining filter or by incorporating sterilizing agents in the form of sterile solid compositions, which can be dissolved or dispersed in sterile water or other sterile injectable medium just prior to use.

Dosage forms for topical administration include powders, sprays, ointments and inhalants. Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

The present invention also provides pharmaceutical compositions that comprise the therapeutic agent and the anti-platelet agent of the present invention formulated together with one or more non-toxic pharmaceutically acceptable carriers. Compounds of the present invention can also be administered in the form of liposomes. As is known in the art, liposomes are generally derived from phospholipids or other lipid substances. Liposomes are formed by mono- or multi-lamellar hydrated liquid crystals, which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together. Methods to form liposomes are known in the art, incorporated herein by reference.

The compounds of the present invention can also be administered to a patient in the form of pharmaceutically acceptable ‘prodrugs.’ The term “pharmaceutically acceptable prodrugs” as used herein represents those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. Prodrugs of the present invention may be rapidly transformed in vivo to the parent compound of the above formula, for example, by hydrolysis in blood. A thorough discussion is provided by Higuchi and Stella.

The combination of the therapeutic agent and the anti-platelet agent can be used to treat diseases and disorders in a subject including but not limited to cancer, atherosclerosis, ischemia-reperfusion following acute myocardial infarction, Henoch-Schonlein purpura nephritis, immune complex vasculitis, rheumatoid arthritis, arteritis, aneurysm, stroke, cardiomyopathy, hemorrhagic shock, crush injury, multiple organ failure, hypovolemic shock and intestinal ischemia, transplant rejection, cardiac Surgery, PTCA, spontaneous abortion, neuronal injury, spinal cord injury, myasthenia gravis, Huntington's disease, amyotrophic lateral sclerosis, multiple sclerosis, Guillain Bane syndrome, Parkinson's disease, Alzheimer's disease, acute respiratory distress syndrome, asthma, chronic obstructive pulmonary disease, transfusion-related acute lung injury, acute lung injury, Goodpasture's disease, myocardial infarction, post-cardiopulmonary bypass inflammation, cardiopulmonary bypass, septic shock, transplant rejection, xeno transplantation, burn injury, systemic lupus erythematosus, membranous nephritis, Berger's disease, psoriasis, pemphigoid, dermatomyositis, anti-phospholipid syndrome, inflammatory bowel disease, hemodialysis, leukopheresis, plasmapheresis, heparin-induced extracorporeal membrane oxygenation LDL precipitation, extracorporeal membrane oxygenation, macular degeneration, and combinations thereof. The specific disease or disorder will depend on the particular therapeutic agent used.

In a particular aspect of the present invention, the disease or disorder can include cancer and the cancer can be treated by administering an anti-cancer monoclonal antibody that induces monocyte, neutrophil, and platelet activation in combination with an anti-platelet agent, such as a GPIIB/IIIa inhibitor (e.g., abciximab). In this aspect, the anti-cancer monoclonal antibody can be an IgG1 antibody that upregulates ADCC and enhanced killing of the cancer cells.

In another aspect of the present invention the therapeutic agent can be used to treat complement-mediated conditions encompassing CPB; coronary artery diseases, such as myocardial infarction and unstable angina; heart failure; cerebrovascular diseases such as stroke and atrial fibrillation; coronary artery bypass grafts; peripheral vascular disease; thromboembolic complications of prosthetic cardiovascular devices such as heart valves and vascular grafts; and deep vein thrombosis following major orthopedic surgery, major fractures, or abdominal surgery. In this aspect, the therapeutic agent can include an anti-complement antibody or fragment thereof such as anti-P, anti-B, anti-C3/C3b, anti-05, anti-D, anti-C8, anti-C9 antibodies, and anti-05b-9 and the anti-platelet agent can be selected from the group consisting of but not limited to aspirin, ibuprofen, Ticlopidine, Clopidogrel, Cangrelor, AZD6140, ReoPro, Aggrastat, Integrilin, cilastazol, Piroximone, Enoximone, Toborinone, Olprinone, and Dipyridamole

The Examples that follow and are not meant to limit the invention unless otherwise stated in the claims. Changes can be made in the composition, operation, and the method of the present invention described herein without departing from the concept and scope of the invention as defined in the claims.

### EXAMPLES

In the following examples, heparinized whole blood from a donor volunteer was circulated in a PVC tubing at 37° C. to allow complement and cellular activation. It is known that alternative complement pathway is activated when blood comes in contact with the artificial surface of the PVC tubing. As a result, complement is activated to release inflammatory molecules. These inflammatory molecules activate neutrophils, monocytes and platelets. The monoclonal antibody NM4047, inhibits complement activation in vitro in human serum. NM4047 inhibits C3a, C5a, and C5b-9 in whole blood in the tubing loop model. Unexpectedly, the IgG1 did not inhibit the activation of neutrophil, monocytes or platelets. In fact, the IgG1 activated each cell type in a dose dependent manner. We attributed these findings to the Fc region on the IgG1. Removal of Fc domain with Ficin generated an F(ab′)2 molecule which inhibited C3a, C5a, C5b-9. Activation of neutrophils, monocytes, and platelets was also inhibited by the F(ab′)2 confirming that Fc region contributed the increased activation seen by the NM4047.

Nearly similar results were observed with the fully human IgG2 antibody. This antibody inhibited C3a, C5a, and sC5b-9 formation. The monoclonal antibody inhibited the activation of neutrophils and monocytes but activated platelets above control levels. Specific activation of platelets in such case was due to the activation of the FcγRIIa receptors by the IgG2. The IgG2 monoclonal antibody has high affinity for FcγII found on platelets. Platelet activation was measured by the upregulated CD62P on the platelets.

Reopro is known to inhibit platelet activation by binding to GpIIb/IIa receptors. Samples containing both IgG2 and ReoPro inhibited all three cell types including platelets. The novel finding that Fc mediated cellular activation can be inhibited by the GPIIb/IIa antagonist especially by ReoPro and Aggrastat and not by integrillin have never been reported before. Discovery of such findings provides a novel solution to complications that arise when therapeutic antibodies with high ADCC activity are generated for cancer therapy in which case by innocent bystander, cells will be activated via the Fc receptor. In experiments where IgG1 was used similar results were observed. While the Fc region of the IgG1 activated neutrophils, monocytes, and platelets, ReoPro only downregulated the Fc effects of the monoclonal antibody on platelets. In cancer therapy, such effects will be desired since NK cells and monocytes are expected to be involved in cancer killing.

Receptors specific for the Fc moiety of IgG (FcγIIR), expressed on a broad range of hematopoietic cells, (see Table). On human leukocytes, three distinct classes of IgG Fc receptors (FcγR) are currently recognized. FcγRI is expressed constitutively on monocytes and macrophages. FcγRII and Fc-yRIII primarily bind immune complexes. Both FcγRII and Fc'γRIII have been demonstrated on neutrophils, eosinophils, and macrophages. FcγRII is, furthermore expressed on monocytes, B cells, basophils, and platelets. The subclass specificity of Fc˜γRII (on platelets) was previously determined as human (h)IgG1=hIgG3>hIgG2,hIgG4. This indicates that differences in subclass specificity between the various forms and differential expression on distinct cells may have major consequences for antibody effector functions. FcγRII is known to be polymorphic with respect to its interaction with mouse (m) IgG I complexes.

Platelets in general bear the FcγRII receptors and can be activated by IgG(s) that carry the Fc region appropriate for activation. Activation of platelets via both complement and non-complement-mediated triggers are well known. Platelets are activated either by Fc receptors because of the Fc of the antibody or by their contact with artificial surfaces. Platelet activation by a monoclonal antibody is dependent on its interactions with both the platelet Fc receptor and FcγRII, and on the antigen (specific or non-specific) that is recognized by the antibody. For instance, activation of FcγII, which could occur due to the use of therapeutic levels of an antibody (alone or in combination with the antigen), activates platelets.

Platelet activation and dysfunction must often be suppressed in order to prevent abnormal bleeding such as those encountered in bypass surgery and thrombosis. Agents that directly or indirectly affect platelet activations are of significant benefit for the treatment of complement-mediated clinical conditions.

### Example 1

### IgG1 (Nm4047) Blocking Monoclonal Antibody Inhibits C3a, C5a, and sC5b-9 Formation but does not Inhibit Cellular Activation

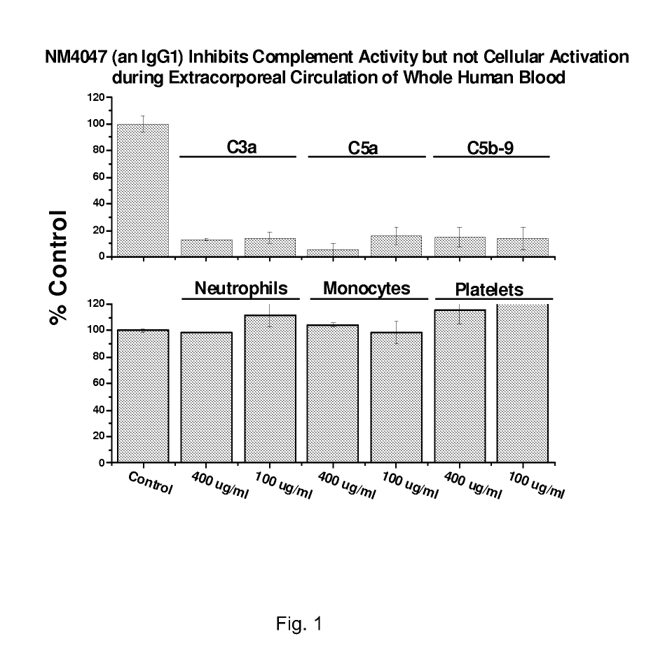

NM4047 is an IgG1 monoclonal antibody that was identified as a blocker of alternative complement pathway activation. The monoclonal antibody was evaluated for inhibition of complement and cellular activation in a whole blood based extracorporeal circulation model. In this model, whole heparinzed human blood was diluted with plasmalyte and circulated in a PVC tubing loop for 2 h at 37° C. to allow complement activation to occur. A total of 21 tubing loops were prepared. The first three loops represented as controls and did not receive the NM4047. Nine tubing loops received 400 μg/ml concentration of NM4047 and the other nine received 100 μg/ml concentration of NM4047. Tubing loops were then rotated for 2 h at 37° C. Following the rotation, aliquots of the blood were stained for flow cytometry for the evaluation of cellular activation. A separate aliquot of blood was centrifuged to separate plasma, which was used for measuring complement activation products in ELISA. As shown in FIG. 1, C3a (Quidel ELISA kit), C5a (Pharmingen), and sC5b-9 (Quidel) were completely inhibited by the monoclonal antibody NM4047. However, activation of neutrophils, monocytes, and platelets was not inhibited. Cellular activation was evaluated using flow cytometry methods. Aliquots of blood sample were stained with FITC labeled CD15 for neutrophils, FITC labeled CD14 for monocytes, and FITC labeled CD61 for Platelets. The cells were also stained with activation markers PE labeled CD11b for neutrophils and monocytes, and PE labeled CD62P for platelets. The staining was done in three groups, one group containing CD15/CD11b, second group contained CD14/CD11b, and the third group contained CD61/CD62P antibodies. Following staining, the cells were lysed with lysis buffer, centrifuged to pellet the cells. After a quick wash with 1% BSA/PBS, the cells were suspended in 1% paraformaldehyde buffer. Cell populations were read in the BD-LSR 1 and Cell Quest software. The raw data taken from Cell Quest were analyzed using Win List software from Verity, populations were measured using Ln Median shifts in the activation marker in the case of neutrophils and monocytes, and population percentage shifts for platelets. The data was then plotted on MicroCal Origin software. FIG. 1 shows that NM4047 does not inhibit cellular activation. The lack of cellular inhibition could only be explained by the fact that the IgG1 had the Fc region. To determine if the predicted Fc is the cause of the observed cellular activation, IgG1 was digested with Ficin protease.

### Example 2

### F(ab)2 Fragment of NM4047 Inhibits C3a, C5a, sC5b-9, and Cellular Activation

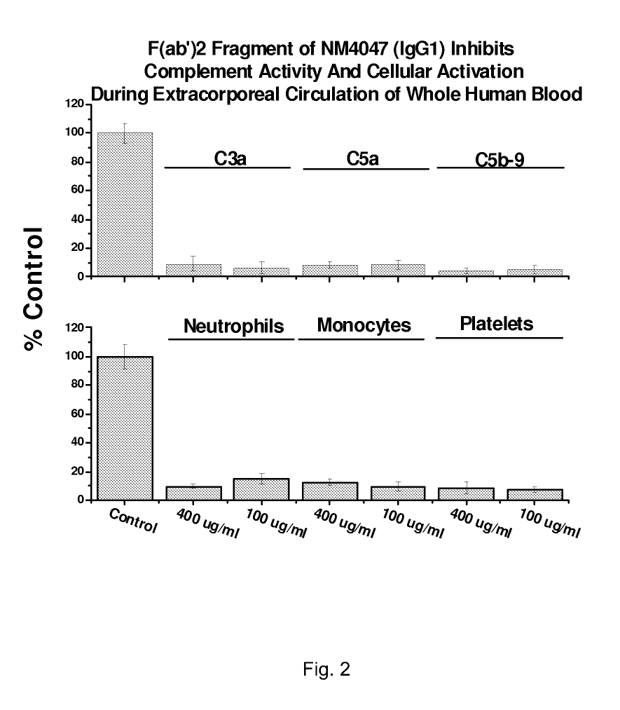

NM4047 was digested with Ficin using standard methods. The F(ab)2 fragment of NM4047 was tested in the extracorporeal tubing loop model explained in Example 1. The whole experiment was run identical to Example 1. Briefly, whole heparinized human blood was diluted with plasmalyte and circulated in a PVC tubing loop for 2 h at 37° C. to allow complement activation to occur. A total of 21 tubing loops were prepared. The first three loops represented as controls and did not receive the NM4047. Nine tubing loops received 400 μg/ml concentration of NM4047 and the other nine received 100 μg/ml concentration of NM4047. Tubing loops were then rotated for 2 h at 37° C. Following the rotation, aliquots of the blood were stained for flow cytometry for the evaluation of cellular activation. A separate aliquot of blood was centrifuged to separate plasma, which was used for measuring complement activation products in ELISA. As shown in FIG. 2, C3a (Quidel ELISA kit), C5a (Pharmingen), and C5b-9 (Quidel) were completely inhibited by the monoclonal antibody NM4047. However, activation of neutrophils, monocytes, and platelets was not inhibited. Cellular activation was evaluated using flow cytometry methods. Aliquots of blood sample were stained with FITC labeled CD15 for neutrophils, FITC labeled CD14 for monocytes, and FITC labeled CD61 for Platelets. The cells were also stained with activation markers PE labeled CD11b for neutrophils and monocytes, and PE labeled CD62P for platelets. The staining was done in three groups, one group containing CD15/CD11b, second group contained CD14/CD11b, and the third group contained CD61/CD62P antibodies. Following staining, the cells were lysed with lysis buffer, centrifuged to pellet the cells. After a quick wash with 1% BSA/PBS, the cells were suspended in 1% paraformaldehyde buffer. Cell populations were read in the BD-LSR 1 and Cell Quest software. The raw data taken from Cell Quest were analyzed using Win List software from Verity, populations were measured using Ln Median shifts in the activation marker in the case of neutrophils and monocytes, and population percentage shifts for platelets. The data was then plotted on MicroCal Origin software. FIG. 2 shows that NM4047 inhibits complement and cellular activation down to the baseline. The fragment F(ab)2 inhibited C3a, C5a, and sC5b-9 formation Inhibition of neutrophils, monocytes, and platelets was also observed. These studies suggest that the lack of inhibition seen in FIG. 1 was solely due to the Fc region of IgG1. The Fc region of the IgG1 can bind Fc receptors on several cells including neutrophils, monocytes, and platelets and could activate these cells. Our goal was to determine of a combination agent added to NM4047 could down regulate the activation of either of the cell types. We tested the effects of various anti-platelet agents to evaluate if a commination therapy can be proposed as a solution to cellular activation by IgG1.

### Example 3

### IgG1 (Nm4047) Blocking Monoclonal Antibody in Combination with Reopro (10 Ug/Ml) Inhibits C3a, C5a, and sC5-9 Formation and Platelet Activation

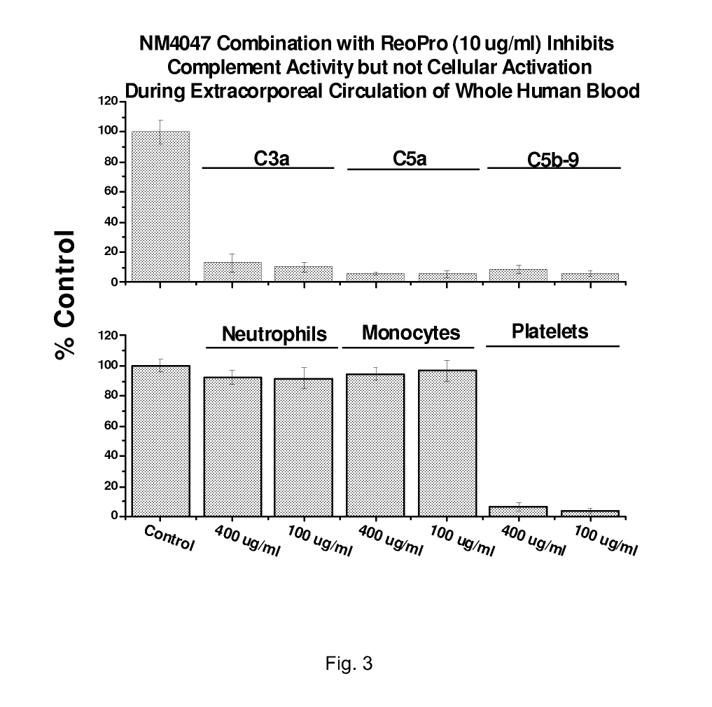

NM4047 (IgG1) monoclonal antibody was evaluated for inhibition of complement and cellular activation in a whole blood based extracorporeal circulation model in the presence of ReoPro. ReoPro is a GPIIbIIIa monoclonal antibody that binds resting and activated platelets. In this experiment, heparinzed human blood was diluted with plasmalyte and circulated in a PVC tubing loop for 2 h at 37° C. to allow complement activation to occur. A total of 21 tubing loops were prepared. The first three loops represented as controls and did not receive the NM4047. Nine tubing loops received 400 μg/ml concentration of NM4047 and the other nine received 100 μg/ml concentration of NM4047. All samples other than controls received 10 μg/ml of ReoPro (clinical material). All tubing loops were then rotated for 2 h at 37° C. Following the rotation, aliquots of the blood were stained for flow cytometry for the evaluation of cellular activation. A separate aliquot of blood was centrifuged to separate plasma, which was used for measuring complement activation products in ELISA. As shown in FIG. 3, C3a (Quidel ELISA kit), C5a (Pharmingen), and sC5b-9 (Quidel) were completely inhibited by the monoclonal antibody NM4047. However, activation of neutrophils, monocytes, and platelets was not inhibited. Cellular activation was evaluated using flow cytometry methods. Aliquots of blood sample were stained with FITC labeled CD15 for neutrophils, FITC labeled CD14 for monocytes, and FITC labeled CD61 for Platelets. The cells were also stained with activation markers PE labeled CD11b for neutrophils and monocytes, and PE labeled CD62P for platelets. The staining was done in three groups, one group containing CD15/CD11b, second group contained CD14/CD11b, and the third group contained CD61/CD62P antibodies. Following staining, the cells were lysed with lysis buffer, centrifuged to pellet the cells. After a quick wash with 1% BSA/PBS, the cells were suspended in 1% paraformaldehyde buffer. Cell populations were read in the BD-LSR 1 and Cell Quest software. The raw data taken from Cell Quest were analyzed using Win List software from Verity, populations were measured using Ln Median shifts in the activation marker in the case of neutrophils and monocytes, and population percentage shifts for platelets. The data was then plotted on MicroCal Origin software. FIG. 3 shows that NM4047 inhibits complement activation. The effect of ReoPro was only visible on platelets and not on neutrophils and monocytes. ReoPro did not affect the level of complement inhibition. ReoPro only effected the platelet activation via the Fc. These unpredictable findings support the idea that ReoPro can block the effect of Fc mediated platelet activation. It is clear that inhibition of platelet activation will inhibit formation of leukocyte—platelet conjugates.

### Example 4

### Dose Dependent Inhibition of Fc-Induced Platelet Activation by Reopro & Aggrastat in Whole Human Blood

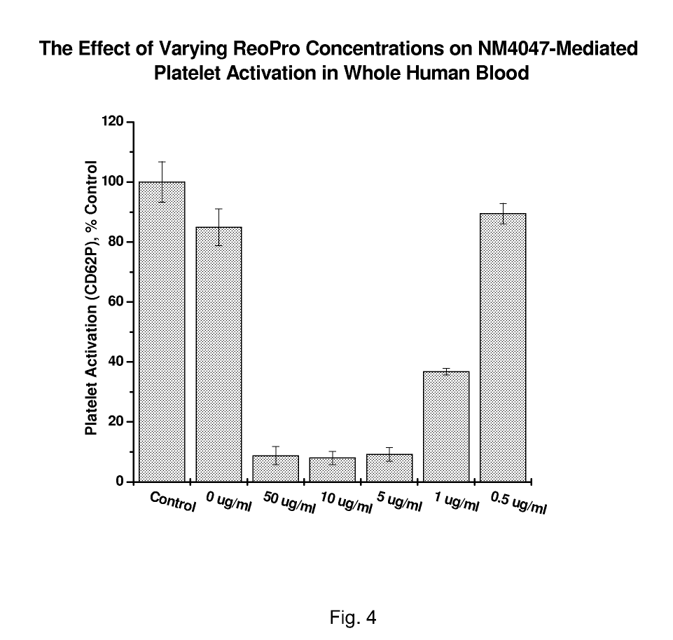

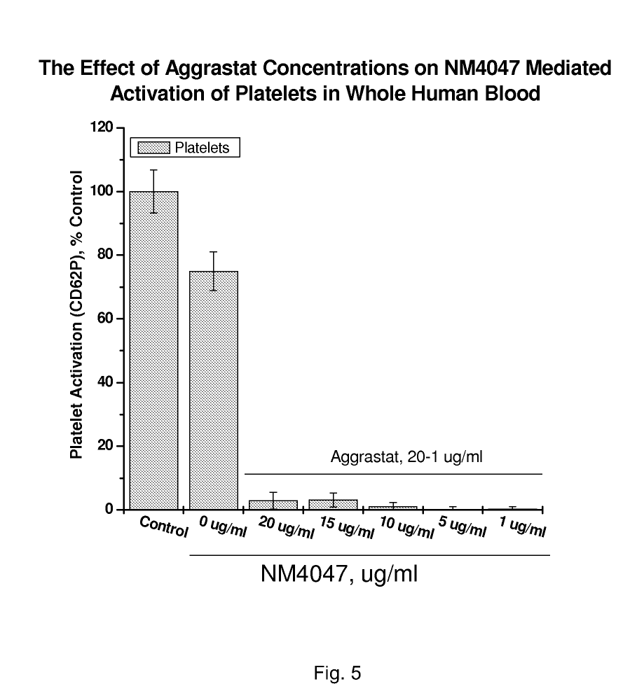

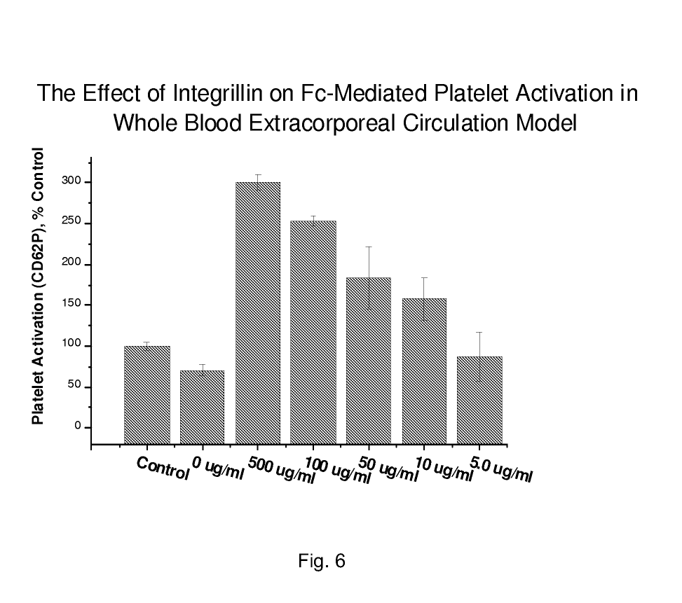

We showed in FIG. 3 that ReoPro is capable of inhibiting the Fc mediated effects of platelet activation at 10 μg/ml. To determine the optimal dose of ReoPro in the tubing loop model, we evaluated the effect of 50 to 0.5 μg/ml concentrations of ReoPro. In all samples including controls, the amount of NM4047 was maintained at 400 μg/ml in blood. The experiment was carried out as in Examples 1-3 except that various concentrations of ReoPro were evaluated in the presence of NM4047. As shown in FIG. 4, ReoPro effect on platelet inhibition was dose dependent with higher concentrations providing maximal inhibition. The effect starts to vanish at 0.5 μg/ml concentration. This concentration range is nearly subtherapeutic and can be used in combination with NM4047 to prevent complement and platelet activation. ReoPro has no effect on neutrophil and monocyte activation. These therapeutic conditions would be more appropriate in situations involving cancer therapy where inhibition of monocytes would not be desired. Therapeutic cancer monoclonal antibodies have an upregulated Fc region, in fact Fc regions in these antibodies are made highly acute for cancer cell killing. Due to an acute Fc, these antibodies can activate platelets depending upon the isoform being used. Combination with ReoPro should prevent platelet activation especially those triggered via the Fc region. As shown in FIG. 4, ReoPro effectively prevented platelet activation. In a separate experiment ReoPro was replaced with Aggrastat, a small molecule based GPIIb/IIa antagonist. As shown in FIG. 5, Aggrastat also inhibited platelet activation in a dose dependent manner with complete inhibition being achieved at 1 μg/ml of aggrastat. Shown in FIG. 6 is the effect of integrillin on platelet activation in the presence of NM4047. As shown, Integrillin, unlike ReoPro, aggrastat and aspirin does not inhibit the activation of platelets. Surprisingly it increased the activation of platelets as measured the CD62P staining. These findings with integrillin are consistent with the literature.

Integrillin does not prevent platelet activation and formation of platelet-leukocyte conjugates. The integrillin only prevents platelet aggregation, which is via the GPIIbIIIa receptor. For combination with NM4047, the selection of anti-platelet agent should be made with care.

### Example 5

### Dose Dependent Inhibition of Fc-Induced Platelet Activation by Aspirin in Whole Human Blood

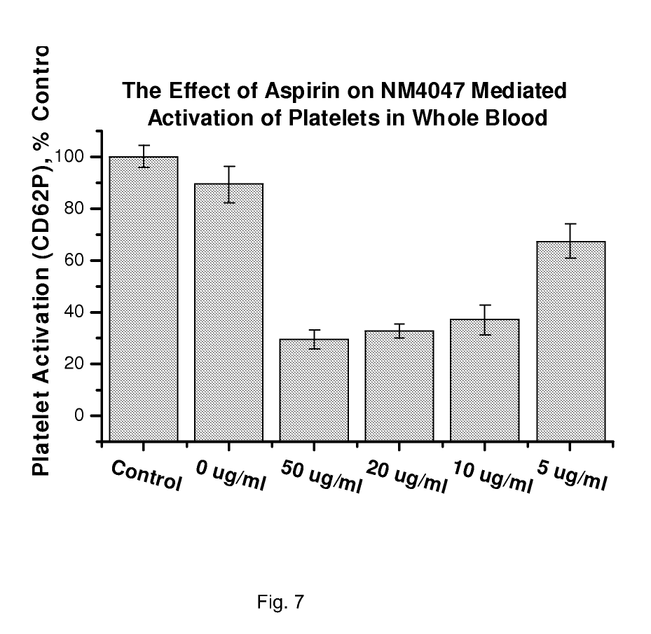

We showed in FIGS. 4 and 5 that both GPIIb/IIa antagonists are capable of inhibiting platelet activation. In this example, we evaluated the effects of Aspirin a commonly used inhibitor of platelet activation. Aspirin is also used as a standard of care for cardiac procedures. However, when tested in combination with NM4047, it did not alter the complement inhibition ability of NM4047, but it prevented platelet activation as shown in FIG. 7. The level of inhibition does not appear to be as robust as shown by ReoPro and Aggrastat. High concentration of Aspirin doses caused hemolysis of erythrocytes. The tubing loop experiments presented in various examples above remained the same. The only difference was in the type of test agent used across these experiments. While good results were noticed with ReoPro and Aggrastat, the cost of such drugs may be prohibitive. In such cases, aspirin may become a viable option.

All references cited herein, including patents, patent applications, papers, textbooks, and the like, and the references cited therein, to the extent that they are not already, are hereby incorporated herein by reference.

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

### Example 6

### Effect of Anti-Platelet Agent on IgG2 Monoclonal Antibody Medicated Cellular Activation

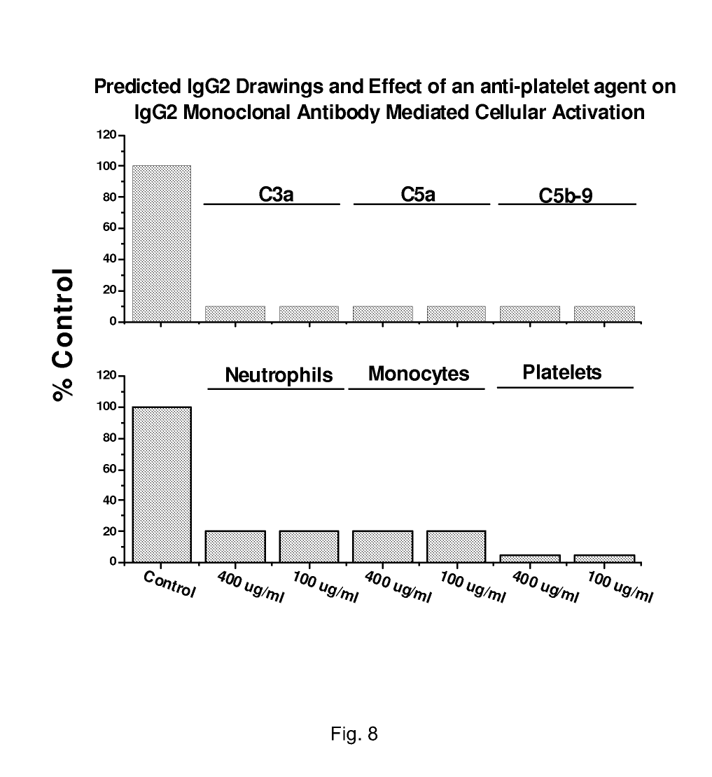

The effect of a blocking complement IgG2 antibody on complement and cellular activation in whole blood is shown in FIG. 8. All complement activationmarkers will be inhibited at a given concentration, which need to be determined using methods well known in the art. In the bottom panel are shown activation of neutrophils, monocytes and platelets. As shown in Table-1, IgG1 binds all three cell types among others. ReoPro only inhibits platelet activation. IgG2 binds and activated only platelets and does not bind neutrophils and monocytes. ReoPro or the selected GPIIb/IIa antagonist can inhibit platelet activation. Therefore, an ideal blocking monoclonal antibody should be an IgG2 we predict the effect of such an antibody would be as shown in FIG. 8. IgG3 will activate neutrophils and monocytes. IgG4 is also a likely choice but could activate monocytes.

1. A method of treating a disorder in a subject, the method comprising: administering to a subject a therapeutic agent that can treat the disorder, the therapeutic agent inducing Fc mediated activation of platelets in the subject; and administering an amount of an anti-platelet agent effective to inhibit the platelet activation.

2. The method of claim 1, the therapeutic agent comprising a monoclonal antibody of the IgG isotype or an antibody fragment comprising an Fc region.

3. (canceled)

4. The method of claim 1, wherein the therapeutic agent comprises at least one of a monoclonal antibody, polyclonal antibody, protein including amino acid sequences of Fc, and fusion protein including amino acid sequences of Fc.

5. The method of claim 4, wherein the therapeutic agent is used to treat at least one of cancer, allograft rejection, HIV, cardiopulmonary bypass, rheumatoid arthritis, stroke, multiple sclerosis, sepsis, autoimmune haematological disorders, complement mediated disorders, or hepatitis B.

6. The method of claim 4, wherein the therapeutic agent is used to treat cancer.

7. The method of claim 4, wherein the therapeutic agent is used to treat a complement mediated disorder.

8. The method of claim 1, wherein the anti-platelet agent comprising a compound that binds to or modulates the GPIIb/IIIa receptor present on platelets.

9. The method of claim 8, wherein the compound comprises a GPIIb/IIIa receptor antagonist that inhibits platelet aggregation.

10. The method of claim 1, wherein the anti-platelet agent inhibits platelet aggregation, leukocyte-platelet aggregation, and platelet CD62P expression.

11. The method of claim 1, wherein the anti-platelet agent inhibits platelet aggregation without inhibiting platelet CD62P expression.

12. The method of claim 1, wherein the anti-platelet agent comprises at least one of a COX 1 inhibitor, a COX 2 inhibitor, an ADP receptor antagonist, a GPIIb/IIIa inhibitor, or a phosphodiesterase inhibitor.

13. The method of claim 12, wherein the anti-platelet agent comprises a GPII/IIIa inhibitor selected from the group consisting of abciximab, eptifibatide, tirofiban, lamifiban, lefradafiban, sibrafiban (Ro-48-3657), orbofiban, xemilofiban, and combinations thereof.

14. The method of claim 12, wherein the anti-platelet agent comprises aspirin.

15. (canceled)

16. The method of claim 12, wherein the anti-platelet agent comprises abciximab.

17-36. (canceled)

37. A method of treating a cancer in a subject, the method comprising: administering to a subject a therapeutic antibody or fragment thereof comprising an Fc sequence, the therapeutic antibody or fragment thereof treating cancer and inducing Fc mediated activation of platelets in the subject; and administering an amount of an anti-platelet agent effective to inhibit platelet activation.

38. The method of claim 37, the therapeutic antibody comprising a monoclonal antibody of the IgGI isotype.

39. The method of claim 37, wherein the anti-platelet agent comprising a compound that binds to or modulates the GPIIb/IIIa receptor present on platelets.

40. The method of claim 39, wherein the compound comprises a GPIIb/IIIa receptor antagonist that inhibits platelet aggregation.

41. The method of claim 37, wherein the anti-platelet agent inhibits platelet aggregation without inhibiting platelet CD62P expression.

42. The method of claim 37, wherein the anti-platelet agent comprises at least one of a COX 1 inhibitor, a COX 2 inhibitor, an ADP receptor antagonist, a GPIIb/IIIa inhibitor, or a phosphodiesterase inhibitor.

43. The method of claim 42, wherein the anti-platelet agent comprises a GPII/IIIa inhibitor selected from the group consisting of abciximab, eptifibatide, tirofiban, lamifiban, lefradafiban, sibrafiban (Ro-48-3657), orbofiban, xemilofiban, and combinations thereof.

44. The method of claim 41, wherein anti-platelet agent inhibits IgG platelet activation.

45. The method of claim 44, wherein the anti-platelet agent comprises abciximab.

46. The method of claim 37, wherein administration of therapeutic agent and the anti-platelet agent does not inhibit activation of leukocytes and NK cells.

47. A method of treating a complement mediated disorder in a subject, the method comprising: administering to a subject a therapeutic antibody or fragment thereof comprising an Fc sequence, the therapeutic antibody or fragment thereof inhibiting complement activation and inducing Fc mediated activation of platelets in the subject; and administering an amount of an anti-platelet agent effective to inhibit platelet activation.

48. The method of claim 47, the therapeutic antibody comprising a monoclonal antibody of the IgG isotype or an antibody fragment.

49. The method of claim 47, wherein the anti-platelet agent comprising a compound that binds to or modulates the GPIIb/IIIa receptor present on platelets.

50. The method of claim 49, wherein the compound comprises a GPIIb/IIIa receptor antagonist that inhibits platelet aggregation.

51. The method of claim 47, wherein the anti-platelet agent inhibits platelet aggregation, leukocyte-platelet aggregation, and platelet CD62P expression.

52. The method of claim 47, wherein the anti-platelet agent inhibits platelet aggregation without inhibiting platelet CD62P expression.

53. The method of claim 47, wherein the anti-platelet agent comprises at least one of a COX 1 inhibitor, a COX 2 inhibitor, an ADP receptor antagonist, a GPIIb/IIIa inhibitor, or a phosphodiesterase inhibitor.

54. The method of claim 53, wherein the anti-platelet agent comprises a GPII/IIIa inhibitor selected from the group consisting of abciximab, eptifibatide, tirofiban, lamifiban, lefradafiban, sibrafiban (Ro-48-3657), orbofiban, xemilofiban, and combinations thereof.

55. The method of claim 47, wherein anti-platelet agent inhibits IgG platelet activation.

56. The method of claim 47, wherein the anti-platelet agent comprises abciximab.

57-75. (canceled)
